(19) United States
(12) Patent Application Publication
(10) Pub. No.: US 2022/0319674 A1
(43) Pub. Date: Oct. 6, 2022

(54) Consultation Assistant For Aesthetic Medical Procedures

(71) Applicant: Crisalix S.A., Lausanne (CH)

(72) Inventors: Jaime Garcia Giraldez, Troistorrents (CH); Fabian Wyss, Dubai (AE)

(21) Appl. No.: 17/597,916

(22) PCT Filed: Jul. 30, 2020
(86) PCT No.: PCT/EP2020/071546

§ 371 Date: Jan. 28, 2022

Related U.S. Application Data
(60) Provisional application No. 62/880,917, filed on Jul. 31, 2019.

(51) Int. Cl. G16H 30/40; G06Q 50/00

(52) U.S. Cl. CPC G16H 30/40; G06Q 50/01

(57) ABSTRACT

A computer-implemented consultation assistant assists in the planning and documenting of aesthetic medical procedures. A modification suggestion component automatically suggests modifications and aesthetic procedures to the patient based on reference imagery of a desired outcome, enabling a patient to explain the outcome they want by using visual references. Also included is an imagery guidance component that instructs a user to record standardised imagery of the patient before the procedure and on one or more occasions after the procedure has been performed. The imagery is automatically aligned in space and video is automatically aligned in time, with colour correction and other processing techniques, to enable a true assessment of the results of the procedure.

### TECHNICAL FIELD

The present teaching relates to computer hardware and software tools, systems and methods for assisting in the planning and documenting of aesthetic medical procedures. In particular, the present teaching relates to tools to aid in planning a procedure based on a desired outcome, and for improving before-and-after video and photography of a procedure.

### BACKGROUND

Aesthetic medical procedures (hereafter, “procedures”), including cosmetic surgery, reconstructive surgery, and non-surgical or minimally invasive procedures such as the use of dermal fillers, are potentially life-changing events. A great deal of effort and expertise are required to plan procedures to achieve a desired outcome, and to inform and manage expectations.

Software tools to help plan and visualise procedures have become an important part of aesthetic medicine. For example, “Development and Implementation of a Web-Enabled 3D Consultation Tool for Breast Augmentation Surgery Based on 3D-Image Reconstruction of 2D Pictures” (Ciechomski et al, J Med Internet Res 2012; 14(1):e21) and WO2014/122253 describe a software tool for generating 3D simulations that enables rapid and accurate pre-visualisation of the outcome of procedures.

Taking full advantage of such tools can be difficult. A patient may not be able to clearly explain what outcome they want to achieve, leading to a time-consuming trial-and-error planning stage where different products are selected, and their effects simulated until the desired outcome is found. There is therefore a need for improved tools for the patient to convey their wishes and for quickly identifying a procedure or procedures that will achieve the desired outcome.

Additionally, some procedures may have only a subtle effect on the patient. To avoid disappointment after a procedure has been performed, careful before-and-after imagery is necessary to show the effect of the procedure. Patients and medical professionals often lack the necessary skills to take quality before-and-after photos or videos, so improved tools for guiding them in this task are needed.

### SUMMARY

In one aspect, the present teaching provides a computer implemented method of selecting an aesthetic procedure to be performed on a patient. The method starts with the step of receiving patient imagery showing a current appearance of at least a target body part of the patient. For example, the desired outcome of the aesthetic procedure may generally be adding volume to the lips, and the patient imagery comprises one or more up-to-date photos or optionally live video showing the patient's face. Or the desired outcome of the aesthetic procedure may generally be a breast enhancement, and the patient imagery therefore shows the patient's torso.

The method continues to the step of receiving reference imagery showing a desired appearance of the target body part. For example, the reference imagery is one or more photos or videos of how the patient wants to look after the procedure.

The method continues with the step of automatically determining a modification to the target body part of the patient that would result in the desired appearance of the target body part. The modification is, for example, a simulated physical change in the appearance of the target body part of the patient so that it looks like the body part in reference imagery.

The method continues with the step of automatically identifying at least one aesthetic procedure that would achieve the modification, which is then presented to a user for selection. For example, where the desired outcome is adding volume to the lips, the identified aesthetic procedure could comprise a specified quantity and/or brand of dermal filler and an indication of injection points. For breast enhancement, the identified aesthetic procedure could comprise a specified implant and position for the implant, and identify a suitable medical professional to perform the procedure.

It will of course be recognised that the non-limiting examples given above are provided to aid understanding and that the method of this first aspect may be used to suggest a wide range of different procedures on many target body parts. Example embodiments of the present teaching are therefore able to analyse a patient to automatically create simulations of modifications to their body, based on pictures or video of the patient's younger self, or of celebrities, friends or relatives, it can automatically suggest treatments and operations and show body modification simulations to the patient or other user such as a medical professional who will perform the aesthetic procedure.

In one embodiment, the patient imagery comprises video of the patient and the modification is overlaid onto the video and viewable by a user. For example, the display may be an augmented reality view overlaid onto live video of the patient. This enables the user to readily assess the simulated modification and decide if it is the outcome they want.

In one embodiment, automatically determining a modification to the target body part comprises: automatically creating a base 3D model of the current appearance of at least the target body part of the patient using the patient imagery; automatically creating a target 3D model of the desired appearance of at least the target body part using the reference imagery; and automatically creating a modified 3D model of at least the target body part of the patient by modifying the base 3D model using the target 3D model. Using 3D models allows permits accurate simulation and presentation of the modification to a user on either a simple 2D display or in 3D, virtual reality or augmented reality displays.

In one embodiment the method further comprises, in response to receiving a selection of an aesthetic procedure from the presented at least one aesthetic procedures, providing information about the selected aesthetic procedure. For example, the user may be provided with educational information about the procedure and its risks and benefits to help ensure that a patient provides informed consent prior to undergoing the procedure.

In a related aspect, the present teaching provides a computer system for selecting an aesthetic procedure to be performed on a patient comprising: an interface for receiving patient imagery showing a current appearance of at least a target body part of the patient, and for receiving reference imagery showing a desired appearance of the target body part; a modification suggestion component that, using the patient imagery and reference imagery, automatically determines a modification to the target body part of the patient that would result in the desired appearance of the target body part; and a procedure selection component that automatically identifies at least one aesthetic procedure that would achieve the modification; wherein the interface presents the one or more aesthetic procedures to a user for selection. Advantages and example embodiments of this aspect of the present teaching will be clear from the prior discussion of the similar method.

In another aspect, the present teaching provides a computer implemented method of documenting an aesthetic procedure performed on a body part of a patient. The method starts with the step of, before performing the procedure, receiving a selection of an action to be performed by the patient using the body part. For example, the action might be a gesture or movement if the body part is part of the torso, arms, legs or glutes. Or the action might be an expression if the body part is part of the face such as the lips, nose, cheeks or eyes.

The method continues with the step of presenting predetermined instructions that direct the patient to perform the selected action and that guide in recording imagery of the patient as they perform the selected action, then recording imagery of the patient performing the selected action before the procedure to obtain “before” imagery. For example, the instructions might include audio visual directions for the patient to perform the action at a specified time or for a specified duration so that the action can be captured in a standardised way. The instructions might also include audio and visual guides for positioning the body part within a field of view of a camera or for adjusting lighting and exposure to obtain a repeatable recording. The imagery of the patient, or at least the target body part is stored and multiple recordings of the patient performing different selected actions can be made to document the patient's appearance before the procedure.

The method continues after performing the procedure with the step of presenting the same predetermined instructions and recording imagery of the patient performing the selected action after the procedure to obtain “after” imagery. By repeating the same instructions before and after performing the procedure, easily comparable, standardised imagery can be recorded and stored to document the outcome of the procedure. Recording the same standardised imagery may be repeated multiple times after the procedure to document changes to the patient over time.

The method optionally continues with the step of automatically aligning the “before” imagery and the “after” imagery to obtain aligned imagery, and then presenting the aligned imagery to a user. Because the imagery has been recorded using a standardised process, it is possible to align it in space and to align video in time so that the action that was performed by the patient can be directly compared. Other alignments can be performed including matching exposure settings, colour balance and similar image features.

In one preferred embodiment, presenting the aligned imagery to a user may comprises presenting the “before” imagery and the “after” imagery side by side or in any other physical relationship or configuration that enables easy comparison. A user may also zoom in on a selected portion of one of the “before” imagery and the “after” imagery, to focus on an area of particular interest, and the same zoom is applied to the other of the “before” imagery and the “after” imagery.

In one preferred embodiment, a user is able to automatically share the aligned imagery on a social media website over the Internet, with the simple click or tap of a suitable share button. The aligned imagery may be automatically processed to add branding or other identifying information prior to sharing.

In a related aspect, the present inventio provides a computer system for documenting an aesthetic procedure performed on a body part of a patient, comprising: a database containing predetermined instructions for each of a plurality of actions that may be performed by the patient using the body part, the instructions for directing a patient to perform the associated action and for guiding in recording imagery of the patient as they perform the associated action; an interface for receiving a selection of an action and for presenting the predetermined instructions associated with the selected action; a camera for recording imagery of the patient performing the selected action; and a storage for storing recorded imagery; wherein the system is used by a user before the aesthetic procedure is performed to receive a selection of an action, to present the predetermined instructions associated with the selected action, and to record imagery of the patient performing the selected action following the instructions to obtain “before” imagery, the storage storing the “before” imagery; and is used by a user after the aesthetic procedure is performed to present the same predetermined instructions associated with the selected action and to record imagery of the patient performing the selected action following the instructions to obtain “after” imagery. Optionally, the system automatically aligns the “before” imagery and the “after” imagery to obtain aligned imagery and presents the aligned imagery to a user via the interface. Advantages and example embodiments of this aspect of the present teaching will be clear from the prior discussion of the similar method.

It will be understood that the computer systems and methods described above can be implemented using one or more pieces of software operating on a single piece of hardware or distributed over multiple pieces of hardware connected over a network such as the Internet. Software embodiments of the present teaching may be stored on a computer readable medium or transferred digitally over a network and executed on a user's hardware such as a personal computer, tablet computer or other mobile device. Embodiments of the present teaching may also run on a central server and users provide inputs and view system outputs via a suitable interface such as a web browser program.

### BRIEF DESCRIPTION OF THE DRAWINGS

Preferred embodiments of the present teaching and examples to aid understanding will be described with reference to the accompanying drawings, in which:

FIG. 1 illustrates a consultation assistant system;

FIG. 2 illustrates steps in planning and simulating a procedure;

FIG. 3 illustrates an interaction between a user and a guided imaging software tool on a tablet computer; and

FIG. 4 illustrates steps in guided recording of before and after imagery of a procedure.

### DETAILED DESCRIPTION

Embodiments of the present teaching can be implemented as part of a combination of software and hardware providing a consultation assistant system.

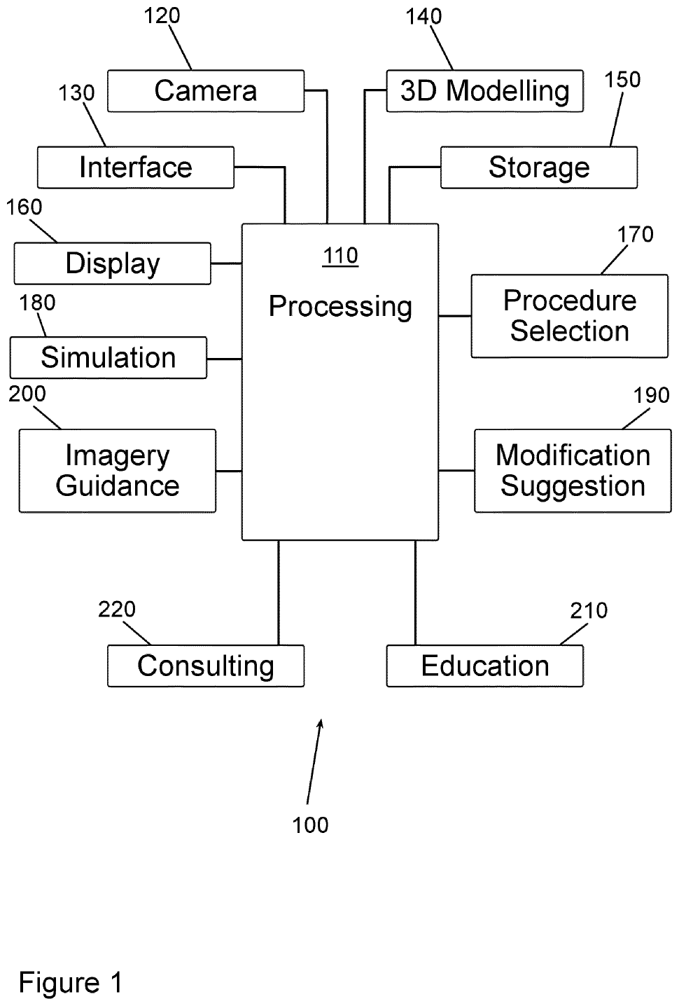

An example consultation assistant system 100 is illustrated in FIG. 1. Components are indicated by interconnected blocks to conceptually represent one possible architecture. These blocks may represent combinations of hardware and software, databases, etc., and may be implemented on one device or distributed over several devices connected via a network such as the Internet. For example, a user interface may run on a mobile device such as a tablet computer or smartphone. Data received via the interface may be processed locally on the mobile device, or the mobile device may communicate with one or more separate server computers which process the received data to provide consultation assistance. Processing of the data may include generating 3D simulations and pre-visualisations of a proposed procedure, recommending procedures, and performing other tasks as explained in more detail below. Simulations and other consultation assistance may be sent back to the initial mobile device for display to the end-user, or to any other display device or storage system.

A processing component 110 of the consultation assistant system 100 receives inputs from a user, manages the generation of simulations and other tasks, and provides outputs back to the initiating user or to a different user. User inputs include patient imagery received via a camera 120, the imagery comprising either or both of still images and video of a patient. Potential users of the system include the patient themselves, or medical professional or even friends or family assisting the patient.

The processing component 110 may use Artificial Intelligence when analysing and processing user inputs and when making recommendations based on those inputs. The AI systems may have access to other data to process user inputs and may use received user inputs to train the AI on an ongoing basis. Other inputs and control of consultation assistant system are provided via a suitable interface 130.

Imagery received from the camera 120 is used by a 3D modelling component 140 to create a base model in 3D of at least those parts of the patient's body that will be affected by a proposed aesthetic procedure. For example, the procedure may be performed on any of the patient's face, breasts, belly, glutes or legs and the 3D modelling component 140 creates a base model of at least that part of the patient. The base model may be stored in a storage 150 and displayed to a user on a display 160. The display may be a 2D or 3D display, a holographic display, or a virtual reality headset, for example. The base model and other processed data may be available to the patient, a medical professional, or any other authorised person with access rights.

A procedure selection component 170 enables a user to select and plan an aesthetic procedure to be performed on the patient. The procedure selection component 170 stores details of a wide range of procedures that can be performed on different parts of the body in a suitable database. These details may include different products and services offered by individual medical professionals, and associated costs. This enables selection of a procedure based on either broad selection criteria or on criteria fine-tuned to a specific product as used by a specific medical professional and within a patient's indicated budget.

A simulation component 180 automatically simulates the results of the selected procedure and creates a modified 3D model of the patient to enable pre-visualisation of the outcome of the procedure. The simulation component 180 can use artificial intelligence (AI) to perform the simulation, the AI using either or both of theoretical models and real-world outcomes of previously performed procedures to improve accuracy in the simulations. For example, the AI may be trained using before and after imagery of previous procedures, including data on specific products used and the individual medical professionals who performed the previous procedures. Simulations may also be run in reverse, where a desired outcome or modification to the patient is provided to the simulation component and a procedure or procedures that will obtain that outcome are automatically identified.

The simulation component 180 may also generate simulations of changes to the patient's body over time, demonstrating the normal effects of aging and the effect of performing different one-off or ongoing rejuvenation procedures. This aids in planning a procedure with a long-term view as well as providing an educational tool for the patient.

To further aid in planning a procedure, a modification suggestion component 190 uses artificial intelligence to automatically create modifications to the patient based on reference imagery of a desired outcome. As will be explained in more detail below, the modification suggestion component 190 assists in the planning of a procedure by enabling the use of visual references to enable the patient to explain what they want.

An imagery guidance component 200 guides the user to record imagery of the patient before the procedure and on one or more occasions after the procedure has been performed. The imagery may be in the form of either or both photos and videos. The imagery is automatically aligned in space and video is automatically aligned in time, with colour correction and other imagery processing techniques to enable a true assessment of the results of the procedure, and to compare the before and after imagery in a standardized way.

An education component 210 educates and informs the patient about selected procedures. A record of educational materials provided to the patient is kept as evidence that the patient was informed and educated about the potential risks associated with a selected procedure. Other caveats make be explained to the patient such as clarifying that no prediction or simulation of a procedure can be 100% accurate. In this way, some of the medical evaluations that are usually required before performing a procedure may be complied with partly or completely without the direct involvement of a medical professional.

A consulting component 220 enables the manual or automatic selection of a medical professional to perform a selected procedure. The patient's personal and medical information, the selected procedure and other data generated from the different components of the consultation assistant system 100 are transmitted to the selected medical professional with the patient's authorisation. A face-to-face consultation can be arranged for a later date, or a consultation can be carried out by video conference via the interface 130 on the patient's computing device. The consulting component 220 may also perform tasks such as (a) estimating a cost of a procedure, (b) providing a payment and ordering system to enable a patient or medical professional to order products or samples, or to pay for the procedure or for use of the consultation assistant system 100, and (c) enabling patients to read and write reviews of their experiences with a medical professional.

The consultation assistant system 100 therefore enables patients to investigate and plan a procedure without the direct help of a professional, but to obtain educational materials and professional help when it is needed. Meanwhile, medical professionals are able to engage with patients having a better understanding of their wishes and are more easily able to make recommendations and demonstrate the likely outcome of those recommendations.

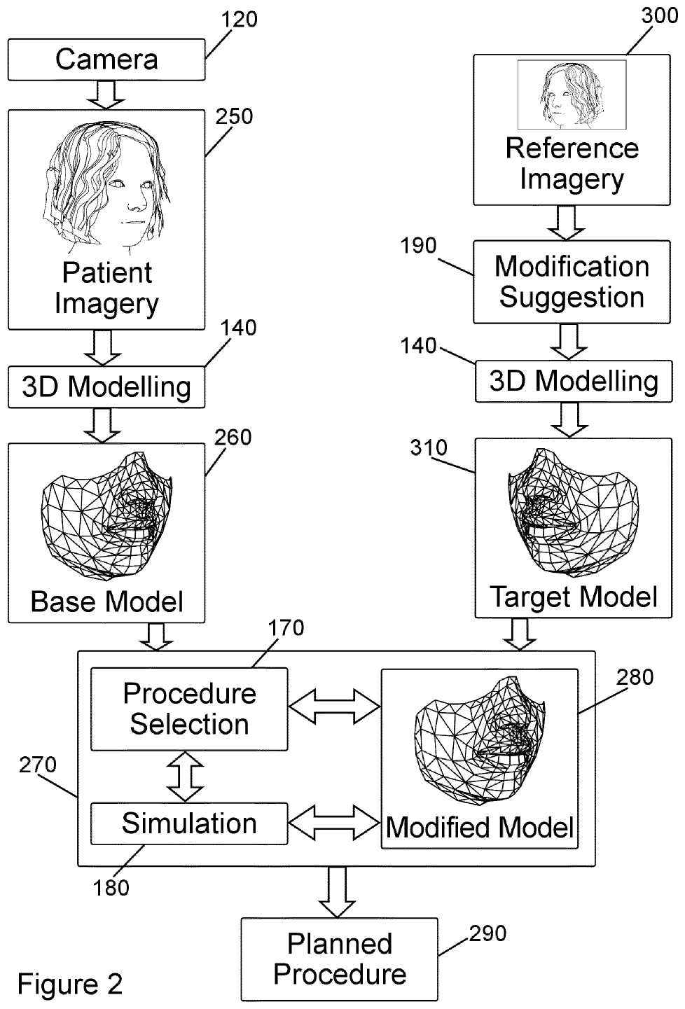

The steps involved in planning and simulating a procedure are shown in more detail in FIG. 2.

In a process similar to the one described in WO2014/122253, a camera 120 is used to capture patient imagery 250. The patient imagery 250 can be still images, video or frames extracted from a video. The imagery is used by the 3D modelling component 140 to create a base model 260 in 3D of the patient or at least those parts of the patient that will be affected by the intended procedure. For example, if it has already been decided that the procedure is to be the injection of dermal fillers into the lips, the base model 260 can be limited to just the lips and surrounding areas of the face as illustrated in FIG. 2

The base model 260 may be any 3D model suitable for realistic simulation such as a voxel-based model as described in Ciechomski et al, or a polygon mesh-based model or some combination of the two. For ease of use, the base model 260 is generated automatically from 2D imagery without the need for the patient's measurements to be input into the system or a detailed 3D scan. Instead, measurements and proportions are calculated automatically from the received patient imagery 250. The base model 260 is textured using the received imagery for near or actual photorealism.

A planning phase 270 includes the steps of procedure selection 170, simulation 180 and generating a modified model 280 which may be performed iteratively until a desired result is achieved.

In one simple workflow, a procedure is selected using the procedure selection component 170. For example, the selected procedure may comprise a specified quantity of dermal filler and desired injection points, though it will be understood that a wide range of different procedures performed on any part of the body may be selected. The outcome of the selected procedure is simulated using the simulation component 180. The simulation component 180 uses the base model 260 and generates a modified model 280 of the patient in 3D.

The modified model 280 may be displayed to a user in any suitable manner but is most effectively displayed using an augmented reality system where the modified model 280 is overlaid onto a live or recorded video of the patient received from the camera 120. Movements of the patient are tracked, and the position and orientation of the modified model 280 updated in response so that the patient can pre-visualise the simulated effects of the selected procedure naturally simply by moving their own body. If the user is happy with the expected result, the planned procedure 290 can be output from the system and used for further consultation.

In an alternative workflow of the planning phase 270, a modified model 280 is created to represent a desired result. The simulation component 180 performs a simulation in reverse to automatically identify and select a procedure or procedures via the procedure selection component 170 that will achieve that result.

In this workflow, the modified model 280 may be created manually by modifying the base model 260 using 3D editing and modelling tools. The modified model 280 may also be generated automatically by various means. For example, shape and skin transformations may be applied to the base model 260 following a pre-defined or custom set of rules such as desirable face proportions and attractiveness criteria.

In general, the different stages of the planning phase 270 may be performed as often as possible and in any order, applying different manual and automatic selections and modifications to achieve a satisfactory outcome that can be chosen as the planned procedure 290.

The above workflows for the planning phase 270 require the patient to be able to explain what they want or for a user to model a desired outcome in 3D. This can require time-consuming trial-and-error iteration to achieve a satisfactory result. In an alternative workflow, 2D reference imagery 300 is used to visually indicate a desired outcome. For example, the patient provides photos or video of themselves when they were younger, or imagery of other people such as friends, relatives or celebrities that the patient wants to look like.

The reference imagery 300 is scanned in or uploaded to the modification suggestion component 190 and is used to create the modified model 280 that is used during the planning phase 270.

In one example workflow, the modified model 280 is created by first generating a target model 310 in 3D from the reference imagery 300 using the 3D modelling component 140 or similar processes as the 3D modelling component 140 uses to create the base model 260 from the patient imagery 250. In this workflow, however, the target model 310 is not used directly for simulation purposes so may be comparatively simple. Instead, the target model 310 is used as a template or other input for modifying the base model 260 to create the modified model 280. For example, the base model may be morphed or otherwise automatically modified until its surface or shape matches the target model or the selected portions of the target model 310. Those modifications are applied to create the modified model 280.

If required, a portion of the reference imagery 300 or of the target model 310 that are to be used to suggest modifications are selected. For example, the reference imagery 300 may be of the whole of a person's face, but the patient only wishes to have the same or similar lips as in the reference imagery. In this example, the lips in the reference imagery 300 or in the target model 310 are selected. It will nevertheless be understood that the reference imagery 300 and resulting target model 310 may be of any part of the body including, but not limited to, the face, breasts, belly, glutes or legs.

During the planning phase 270, the modified model 280 is conveniently used in a reverse simulation workflow as described above to automatically identify a procedure or procedures that will achieve the desired result. Advantageously, the patient is therefore presented with a planned procedure 290 without having to explain what they want and without going through a time-consuming iterative process of procedure selection 170 and simulation 180 to achieve the desired result.

Patients and medical professionals are generally not skilled at taking “before and after” photos or videos to accurately document or monitor the outcome of a procedure. Many factors have an impact such as lighting, the distance to the patient and their pose, and it is difficult to record imagery that is both naturalistic and readily comparable, especially for subtle interventions.

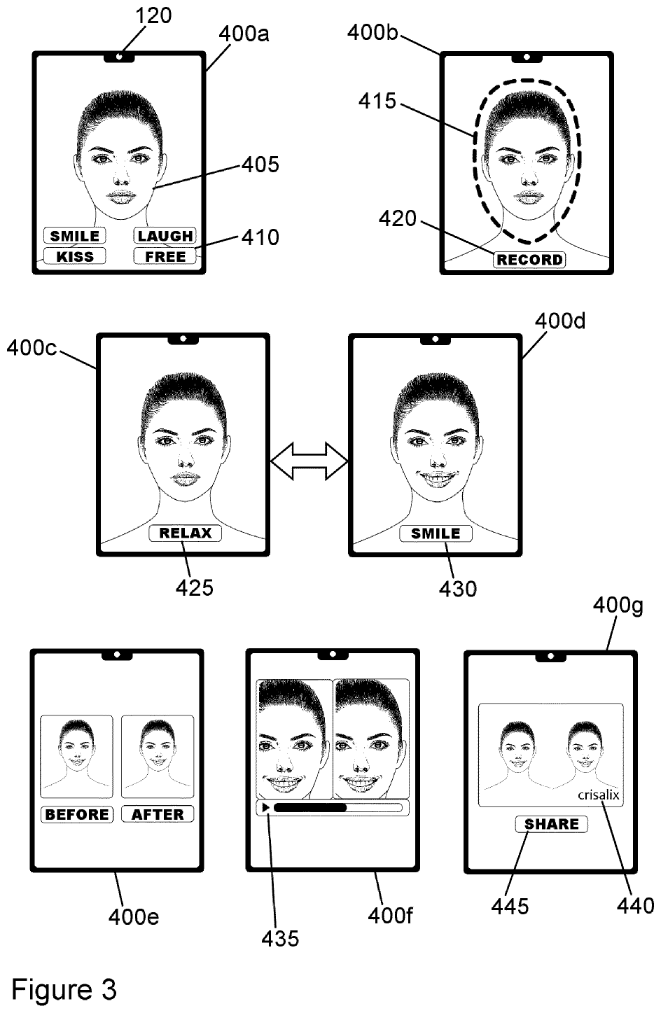

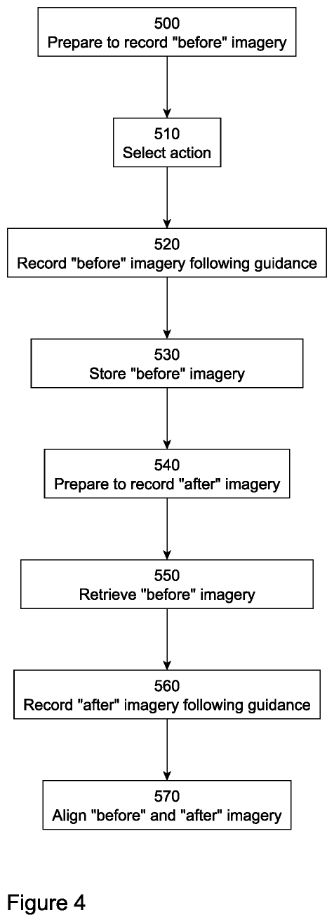

An effective approach to creating easily comparable before and after imagery of the patient using the imagery guidance component 200 is illustrated in FIGS. 3 and 4. FIG. 3 illustrates different views of a tablet computer 400 being used by the patient to record and process their before and after imagery and FIG. 4 is a flowchart representing an example sequence of steps taken by a user. It will of course be recognised that there are many possible variations in the depicted method and that a wide range of different computing or imaging devices may be used.

The imagery guidance component 200 guides the patient, with or without the assistance of a medical professional or other user, to capture standardised imagery such as photos and videos before the procedure through a guided interface. Captured imagery is used as a basis to visually guide the capture of further imagery at any time or at multiple times after the procedure. The captured “after” imagery is automatically aligned with the “before” imagery to enable a true assessment of the results of the procedure.

To start, the user or users prepare to record the “before” imagery 500 using a suitable camera. The patient may be the only user, taking imagery of themselves, or the patient may have the assistance of another person such as a medical professional. The camera may be capable of recording photos or videos such as the camera 120 on a handheld device such as a smartphone or tablet computer 400 or a webcam. For convenience, the camera is on the same device that is running the imagery guidance component 200 or is connected to that device via a network.

Next, using an interface of the imagery guidance component 200, the user selects an action from a provided list 510. Naturalistic imagery is easier to capture if the patient is performing a comfortable, repeatable action, rather than taking up a forced, static position. The list of actions includes expressions or gestures or poses depending upon the part of the body affected by the procedure. For example, if the procedure affects the face, the list of actions may include expressions such as smiling, kissing, laughing, frowning, raising eyebrows or a “free” expression where the patient chooses their own expression. For example, tablet computer 400a shows a potential interface screen displaying the user's face 405 as captured via the inbuilt camera 120 and buttons 410 for selecting different expressions. If the procedure affects other parts of the body, the list of actions may include gestures such as stretching or performing a favourite dance move.

Once an action is selected, the user records the “before” imagery following guidance 520 received from the imagery guidance component 200. The guidance may include directions for performing the selected action and guidance for recording the selected action.

As shown on tablet computer 400b, recording guidance may include visual cues 415 for positioning the camera and patient, such as having the patient face on or at a specified angle to the camera and centering the patient in the image. Audio cues such as verbal instructions or other cues to indicate correct positioning and alignment may also be used. The imagery guidance component 200 may receive and automatically analyse a live preview from the camera and provide responsive guidance such as suggesting moving to an area with more or less light if the imagery is poorly exposed. The user then presses a record button 420 to take a photo or sequence or photos or to start recording video imagery.

As shown on tablet computers 400c and 400d, instructions for performing the selected action may include visual cues telling the patient to relax 425, then directing them to perform the selected action, such as smiling 430, and then directing the patient to return to a relaxed pose or expression after a suitable time has elapsed. A timed sequence of the patient performing the selected action is therefore recorded. The instructions may include either or both of audio cues and verbal direction.

The recording of the action may be repeated as often as necessary, at the request of the user, for example, or if the imagery guidance component 200 detects that the patient or camera has moved out of position. In this way, one or more videos or still images of the patient performing the selected action at a standardised rate or over a standardised time frame are recorded.

The recorded “before” imagery is then stored 530, either locally on the device or on a server computer or both. The imagery is stored along with a description of or a reference to the selected action and/or the guidance that was used to record the imagery so that the selected action can be repeated at a later time. Other information such as time of day or location may be automatically or manually included to help recreate the environment in which the “before” imagery was recorded.

The steps of selecting an action 510 and recording the “before” imagery 520 are repeated as often as desired to obtain a detailed record of the patient before the procedure.

At any time after the procedure, the user prepares to record the “after” imagery 540. They retrieve the “before” imagery from storage 550 and optionally attempt to recreate the environment and lighting. The user then selects one of the previously recorded actions and records the “after” imagery following the same, standardised recording and performance instructions 570 in order to recreate the same actions, expressions and poses as recorded in the “before” imagery. In this way, the before and after imagery have both been created using a standardised approach. This alone enables the creation of significantly better and more readily comparable before and after imagery, which better captures the results of the procedure.

Even more advantageously, the imagery guidance component 200 also automatically aligns the “before” and “after” imagery 570 for a given action. Alignment of the imagery can be achieved using any combination of different processes such as aligning the patient in position or scale in the imagery, aligning in time of videos, and aligning lighting/exposure and colour to match imagery taken in different environments.

For example, the scale and cropping of the imagery can be adjusted so that the patient is the same size and in the same portion of the frame in both the before and after imagery. The timing of videos can be aligned so that a guided action starts at the same time such that actions performed following the standardised guidance are synchronised. Optionally, the user is also able to manually adjust alignment to correct for further slight mismatches not compensated for automatically if, for example, the patient did not follow the guidance precisely or in the same way.

The before and after imagery can be displayed in any desired composition, such as side by side as illustrated on tablet computer 400e. A desired portion or close up of either of the before and after imagery can be selected and the display of the other imagery automatically zoomed to the same portion, as illustrated on tablet computer 400f. Automatic transitions can be added and video playback controls 435 provided to view the imagery in any desired format and style. These compositions enable even better analysis of the results of a procedure than the guided imagery alone.

Merged imagery, comprising still images or videos, optionally overlaid with suitable branding 440 can be prepared automatically from the aforementioned compositions. As illustrated on tablet computer 400g, the interface enables the user to share 445 this merged imagery on social media or to download it for sending to friends, family or medical professionals outside of the interface.

1. A computer implemented method of documenting an aesthetic procedure performed on a body part of a patient, comprising:
before performing the procedure, receiving a selection of an action to be performed by the patient using the body part;
presenting predetermined instructions that direct the patient to perform the selected action and that guide in recording imagery of the patient as they perform the selected action;
recording imagery of the patient performing the selected action before the procedure to obtain “before” imagery;
after performing the procedure, presenting the same predetermined instructions that direct the patient to perform the selected action and that guide in recording imagery of the patient as they perform the selected action;
recording imagery of the patient performing the selected action after the procedure to obtain “after” imagery;
automatically aligning the “before” imagery and the “after” imagery to obtain aligned imagery;
presenting the aligned imagery to a user.

2. The method of claim 1, wherein receiving a selection of an action includes presenting a list of actions to a user and receiving a selection of an action from the list.

3. The method of claim 2, wherein the body part is a part of the face and the list of actions includes at least two of smile, kiss, laugh, frown, and raise eyebrows.

4. The method of claim 3, wherein the list of actions comprises a free expression option.

5. The method of claim 1, wherein the predetermined instructions comprise a guide displayed on a camera display for guiding a user to position the body part within a field of view of the camera.

6. The method of claim 1, wherein the predetermined instructions include at least one of visual instructions and audio instructions directing the patient to perform the selected action over a predetermined time frame.

7. The method of claim 1, wherein recording imagery comprises recording a video of the patient.

8. The method of claim 1, wherein automatically aligning the imagery comprises automatically performing at least one imagery correction technique on either or both of the “before” imagery and the “after” imagery, the at least one imagery correction technique selected from the list including: colour correction, exposure, level correction, sharpness adjustment, and noise reduction.

9. The method of claim 1, wherein automatically aligning the imagery comprises automatically positioning the body part in the same position in the “before” imagery and in the “after” imagery.

10. The method of claim 1, wherein the imagery is a video and wherein automatically aligning the imagery includes automatically aligning the timing of the patient performing the selected action in the “before” imagery and in the “after” imagery.

11. The method of claim 1, wherein presenting the aligned imagery to a user includes at least one of: presenting the “before” imagery and the “after” imagery side by side, presenting the “before” imagery and the “after” imagery in a selected configuration, and zooming in on a selected portion of both the “before” imagery and the “after” imagery.

12. The method of claim 1, further comprising enabling a user to automatically share the aligned imagery on a social media website over the Internet.

13. A computer system for documenting an aesthetic procedure performed on a body part of a patient, comprising:
a database containing predetermined instructions for each of a plurality of actions that may be performed by the patient using the body part, the instructions for directing a patient to perform the associated action and for guiding in recording imagery of the patient as they perform the associated action;
an interface for receiving a selection of an action and for presenting the predetermined instructions associated with the selected action;
a camera for recording imagery of the patient performing the selected action;
a storage for storing recorded imagery;
wherein the system is used by a user before the aesthetic procedure is performed to receive a selection of an action, to present the predetermined instructions associated with the selected action, and to record imagery of the patient performing the selected action following the instructions to obtain “before” imagery, the storage storing the “before” imagery;
and is used by a user after the aesthetic procedure is performed to present the same predetermined instructions associated with the selected action and to record imagery of the patient performing the selected action following the instructions to obtain “after” imagery;
the system automatically aligning the “before” imagery and the “after” imagery to obtain aligned imagery and presenting the aligned imagery to a user via the interface.

14-27. (canceled)
